(19) United States
(12) Patent Application Publication
(10) Pub. No.: US 2021/0353932 A1
(43) Pub. Date: Nov. 18, 2021

(54) ELECTRONIC TEXTILE BASED RHEUMATOID ARTHRITIS THERAPY GLOVE

(71) Applicant: ISTANBUL TEKNIK UNIVERSITESI, Maslak Sariyer Istanbul (TR)

(72) Inventors: Gozde GONCU BERK, Gumussuyu Beyoolu Istanbul (TR); Nese TOPCUOGLU, Istanbul (TR)

(21) Appl. No.: 16/322,883

(22) PCT Filed: Nov. 27, 2017
(86) PCT No.: PCT/TR17/50611

§ 371 Date: Feb. 1, 2019

(30) Foreign Application Priority Data
Dec. 31, 2016 (TR) 2016/20482

(51) Int. Cl. A61N 1/04; A61N 1/36; A61F 5/01

(52) U.S. Cl. CPC A61N 1/0484; A61F 5/0118; A61N 1/36021

(57) ABSTRACT

The electronic textile based rheumatoid arthritis therapy glove is for treating deformities in finger joints and wrist area caused by rheumatoid arthritis and treating chronic pain caused by rheumatoid arthritis disease.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The invention relates to medical devices and wearable technologies.

The invention particularly relates to an electronic textile based rheumatoid arthritis therapy glove used for treating deformities and managing chronic pain in finger joints and wrist area caused by rheumatoid arthritis.

### 2. Description of Related Art Including Information Disclosed Under 37 CFR 1.97 and 37 CFR 1.98

Rheumatoid arthritis is an autoimmune inflammatory joint disease, which may affect various organs and systems, primarily affecting small joints (hand joints, wrists and ankles) and elbows, shows chronic course (over 6 weeks), and causes damage on the affected joint.

Rheumatoid Arthiritis causes damage on the affected joint and could lead to disability. It is a common disease, which affects one in every 100 people, at some stage of their life. It can develop at any age, but it usually starts at the age of 40-60. It is seen three times more in women than men.

Immune system is a system, which protects us and fights against bacteria, viruses, and all kinds of foreign substances (antigens) that may enter our body. In the case of autoimmune diseases, the immune system also fights against our own body by generating immune serum (antibodies) against some of our tissues. This may sometimes be directed to a particular organ such as the thyroid, liver, or sometimes systemic rather than organ-specific. Therefore, rheumatoid arthritis is one of systemic autoimmune connective tissue diseases.

The cause of this aberration in the immune system is not exactly known. However, the genetic nature of the person and environmental factors (smoking and some infections) may be the cause. The inflammation of the joint and its surroundings causes damage to the joint (cartilage, bone, and ligaments) over time. In the joints that are affected, pain, swelling, increase in joint temperature and movement restriction are encountered. Feeling of stiffness occurs in these joints for at least one hour in the mornings or after resting.

Chronic pain caused by rheumatoid arthritis is treated by using pharmacological treatment and physiotherapy methods. Hot and cold thermotherapy, ultrasound, and electrical stimulation are the most commonly used physiotherapy modalities in the treatment of pain and stiffness due to Rheumatoid Arthritis.

In addition Rheumatoid Arthritis disease is also treated by patient training in joint protection, exercise and occupational therapy, balneotherapy, and with the use of splints and auxiliary devices. Thermotherapy, ultrasound, and electrical stimulation modalities are often used to treat pain and stiffness, while splints and auxiliary devices are used to treat joint deformities and allow patients to perform their activities more comfortably.

According to the article of Prof. Dr. Nuran Turkcapar, December 2015; there is no treatment that would completely eliminate rheumatoid arthritis. However, treatments make a big difference in the reduction of symptoms and prevention of disability. Treatment goals are:

To reduce disease activity as much as possible to prevent joint damage.

To reduce pain and stiffness in joints.

To treat other symptoms of the disease, if present.

To prevent the development of disease-related risks such as cardiovascular disease and osteoporosis.

To reduce disease activity and prevent joint damage

As soon as a person is diagnosed with rheumatoid arthritis, rheumatism medicines that modify the condition of the disease (change the course of the disease) should be started. These drugs are; methotrexate, sulfasalazine, leflunamide, and hydroxychloroquine. Although gold salts have been successfully used in the treatment of rheumatoid arthritis in the past, nowadays their use is quite limited due to their cost and side effects. They are still used in some European countries. Although limited in their use, drugs such as cyclosporine, azathioprine, cyclophosphamide, penicillamine, mycophenolate mofetil are other therapeutic agents that can be used in the case of severe systemic findings.

Nonsteroidal anti-inflammatory drugs (such as naproxen, diclofenac, indomethacin) and low-dose corticosteroids (5-10 mg/day) are used on early stages to reduce pain and inflammation in patients. Generally, a gastroprotective medication is added to the treatment for the side effects of these drugs. These treatments are stopped when the effects of the disease modifying drugs are observed; while sometimes they can be used for short term in sickness exacerbations.

Under all these treatments, patients with rheumatoid arthritis have an increased susceptibility to infections. For this reason, vaccination must be made against preventable diseases. If there is no immunity, hepatitis B and A vaccines, annual influenza vaccine and pneumococcal vaccines (effective once every 5 years or long-acting, lifetime effective) are recommended.

The patient needs to be kept active as much as possible. Both joint range of motion and muscular strength should be protected by regular exercises. Swimming is a good exercise. In the presence of a physiotherapist, the patient should learn exercises that he/she can do on his/her own, and should be able to do these exercises independently.

Corrective surgery can be made by orthopedics (such as hip and knee prostheses) and hand surgery can be performed in some cases with very advanced deformities.

Patients should be acknowledged that they have a long-term illness (lasting for years, perhaps a lifetime). For this reason, education of the patient is very important. The better a person knows about his/her illness, the less likely he/she will be concerned, and the better he/she will learn to get through and live together with the illness.

Also in the prior art, deformities in fingers arising from Rheumatoid Arthritis disease are treated with braces, splints, and arthritis rings.

The United States patent application No. U.S. Pat. No. 4,173,218A encountered in the literature, the patented product consists of a glove-shaped envelope structure and fluid contained within the envelope structure. This structure minimizes the impacts that may occur during hand movements and thus reduces the pain that may occur in the fingers and hands. The fluid is heated and used in Rheumatoid Arthritis therapy, when required. The ventilation zones found in the finger joint regions can provide cooling/refreshment, when required.

Also in the literature, in the American patent application No. U.S. Pat. No. 5,853,210A, a glove structure is disclosed, which facilitates writing for physically disabled hands (such as for people who have rheumatoid arthritis disease). A connecting piece found at the palm part of the glove allows fixing of a pen at the palm.

Also in the literature, in the American patent application No. US2010324616A1, a dressing apparatus with electrical conductivity, which can transmit infrared thermal treatment and electrotherapy to the joints of the patient, and an electrotherapy stimulator device which sends low pulse electrical signal to this apparatus, operates with a battery, and contains in its structure all other required electronic components required for formation of the signal, are disclosed.

However, a wearable electrical stimulation device which directly targets finger joints affected by rheumatoid arthritis disease is not disclosed in the said literature.

As a result of the above-mentioned reasons, a novel electronic textile based rheumatoid arthritis therapy glove is needed for treating deformities and reducing pain caused by rheumatoid arthritis in finger joints and wrist area.

### BRIEF SUMMARY OF THE INVENTION

From the known status of the art, the purpose of the invention is to provide a novel electronic textile based rheumatoid arthritis therapy glove, which eliminates the prior art disadvantages.

Another purpose of the invention is to provide a structure, which eliminates the need for using more than one splint or supportive products for deformities formed at hands due to rheumatoid arthritis disease.

Another purpose of the invention is to provide a structure, which prevents frequent loss of the splint or supportive product used for deformities formed at hands due to rheumatoid arthritis disease.

Another purpose of the invention is to provide a structure, which also allows treatment of finger deformities caused by disease in addition to reducing pain of patients.

Another purpose of the invention is to provide a structure allowing pain management and treatment at home for rheumatoid arthritis disease, which is an autoimmune disease and requires constant rehabilitation.

Another purpose of the invention is to provide a structure, which prevents continuous consumption of pain killers by patients.

Another purpose of the invention is to provide a structure, which improves the quality of life of patients who are in need thereof.

### BRIEF DESCRIPTION OF THE SEVERAL VIEWS OF THE DRAWINGS

FIG. 1 is a representative view of the electronic textile based rheumatoid arthritis therapy glove according to the invention.

### REFERENCE NUMBERS

1. Plastic Immobilization Support

2. Plastic Immobilization Support Pocket

3. Control Unit

4. Textile Electrodes

5. Joint compression and immobilization lamina

6. Signal Transmission Line

7. Signal Transmission Line Insulation

8. Wristlet

9. Wristlet Fixing Apparatus

### DETAILED DESCRIPTION OF THE INVENTION

In this detailed description, the novelty according to the invention is only disclosed for better understanding of the subject without forming any limiting effect.

The invention relates to a therapy glove, which consists of textile electrodes (4) formed by processing of conductive yarns on polyester knitted fabric via optimization of computer-assisted embroidery parameters, a signal transmission line (6), and a control unit (3) attached to this line by metal snap fasteners, and which can perform electrical stimulation.

The invention is an electronic textile based rheumatoid arthritis therapy glove used for treating deformities and managing pain in finger joints and wrist area caused by rheumatoid arthritis, characterized in that; it comprises: a control unit (3) positioned on said glove providing electrical stimulation for the treatment of finger joint pain caused by rheumatoid arthritis disease, and can be attached and detached easily due to its snap fastener structure, textile electrodes (4) transmitting the electrical stimulation signal received from said control unit (3) to the skin of the patient, positioned at the glove posterior and anterior areas such that they would correspond to the points of pain at the finger joints affected from the rheumatoid arthritis disease, and formed by processing of conductive yarns on polyester knitted fabric via optimization of computer-assisted embroidery parameters, a signal transmission line (6) carrying the signal transmitted from the control unit (5) to said textile electrodes (4) and formed by processing on polyester textile surface by using silver coated polyamide conductive yarn.

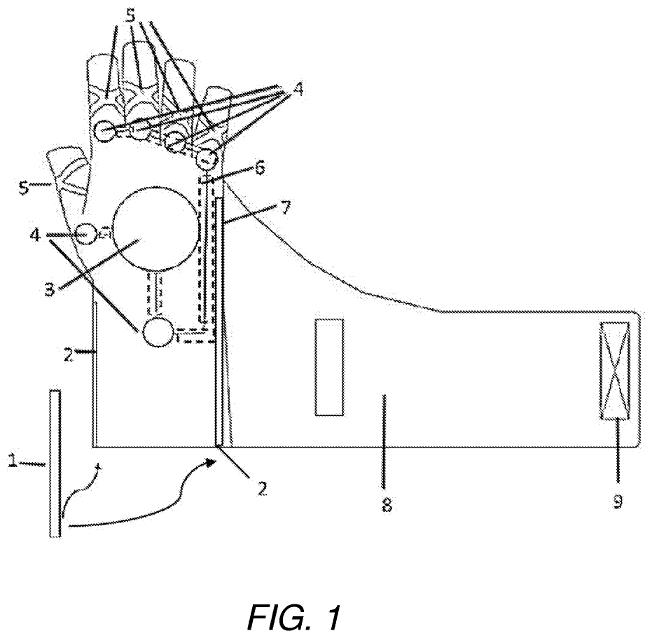

FIG. 1 shows a front view of the electronic textile based rheumatoid arthritis therapy glove (1) according to the invention.

Said electronic textile based rheumatoid arthritis therapy glove consists of the main components of: a plastic immobilization support (1) used for treatment of ulnar deviation caused by rheumatoid arthritis, an immobilization support pocket (2) allowing said immobilization support (1) to be positioned on said glove, a control unit (3) positioned on said glove providing electrical stimulation for the treatment of finger joint pain caused by rheumatoid arthritis disease, and can be attached and detached easily due to its snap fastener structure, textile electrodes (4) transmitting the electrical stimulation signal received from said control unit (3) to the skin of the patient and positioned at the glove anterior and posterior areas such that they would correspond to the points of pain at the finger joints affected from the rheumatoid arthritis disease, a joint compression and immobilization lamina (5) providing immobilization for the treatment of swan-neck and buttonhole (boutonniere) deformations formed at the finger joints due to rheumatoid arthritis disease and positioned at the upper region of the second joints of the fingers in transverse form and at the rear region thereof in straight form, a signal transmission line (6) carrying the signal transmitted from the control unit (3) to said textile electrodes (4) and formed by processing on polyester textile surface by using conductive yarn, a signal transmission line insulation (7) made of thermoplastic membrane material and ensuring electrical insulation of said signal transmission line (6), a wristlet (8) for preventing ulnar deviation and ensuring wrist stabilization by being wrapped around the wrist area of the glove, starting from the little finger tip of the patient, and a wristlet fixing apparatus (9) ensuring fixing of said wristlet (8).

Said plastic immobilization support (1) prevents ulnar deviation caused by rheumatoid arthritis disease by being attached to the plastic immobilization support pocket (2) positioned on said glove, and ensures that the wrist joint stays in a stabilized position.

Said wristlet (8) starts from the tip of the little finger, is wrapped around the wrist part of the glove, and thus also ensures ulnar deviation and wrist stabilization. Said wristlet (8) is fixed by a wristlet fixing apparatus (9).

Said joint compression and immobilization lamina (5) is positioned at the upper region of the second joints of the fingers in transverse form and at the rear region thereof in straight form, and it is formed by lamination of thermoplastic polyurethane membranes with compression characteristics on the polyester textile surface under a certain temperature and pressure. Said joint compression and immobilization lamina (5) allows treatment of swan-neck and buttonhole (boutonniere) deformities formed at the finger joints of the patient.

Said glove comprises 12 textile electrodes (4), 6 at the posterior region and 6 at the anterior region thereof. While the textile electrodes (4) found on said glove are positioned such that they would correspond to the first joints of the fingers and the wrist, the textile electrodes (4) found at the anterior region of the glove are positioned such that they would correspond to the second joints of the fingers and the wrist. Electrical stimulation is applied by means of said textile electrodes (4) to the nerves of said patient, which send messages about pain sensation to the brain. In this way, the pain feeling of the patient is significantly reduced by disrupting the pain signals sent to brain

Said textile electrodes (4) preferably have 15 mm diameter and they are formed by processing silver coated polyamide conductive yarns on the polyester textile surface forming the glove, at certain stitch length and density, by using optimized embroidery parameters.

Said textile electrodes (4) are connected to the control unit (3) by means of the signal transmission line (6). Said signal transmission line (6) connects the textile electrodes (4) found at the upper and inner surfaces of the glove to each other and to the anode and cathode ends of said control unit (3).

Said signal transmission line (6) is formed by processing on polyester textile surface by using silver coated polyamide conductive yarn. Said signal transmission line (6) is designed such that the signal sent from the control unit (3) would be optimized to have the highest SNRdB (Signal Noise Ratio) value.

Said textile electrodes (4) and signal transmission line (6) are designed such that they would transmit low voltage electric signal at certain frequencies and pulse bandwidths to the points of pain at the finger joints affected by rheumatoid arthritis disease.

Said control unit (3) forms a signal in square wave form which can be adjusted at pulse characteristics of 0-80 imA, 2-120 Hz, and 40-260 microseconds for the treatment of chronic pain seen in finger joints due to rheumatoid arthritis disease. The signal transmission line (6) transmitting signal from said control unit (3) to said textile electrodes (4) is coated by thermoplastic membrane and the signal transmission line insulation (7) is provided from the inner and outer surfaces of the textile. Said signal transmission line insulation (7) is positioned so as to provide the highest quality signal transmission.

Said textile electrodes (4) are insulated by the same method only from the outer surface of the glove. Said signal transmission line (6) is connected to the control unit (3) via metal snap fasteners.

The glove according to the invention is washable and washing operation can be performed by removing the control unit (3) from the snap fastener. Said control unit (3) also contains the power source of the system. When required, the control unit (3) can be removed and charged.

1-16. (canceled)

17. An electronic textile based rheumatoid arthritis therapy glove for treatment of deformities in finger joints and wrist area caused by rheumatoid arthritis disease and pain management, the glove comprising:
a control unit comprising at least one easily assemblable and disassemblable snap fastener structure, enabling electrical stimulation for treatment of pain caused by rheumatoid arthritis, and located on said glove; and
a signal transmission line located on inner and outer portions of the glove such that it corresponds to pain points of the finger joints affected by the rheumatoid arthritis disease, transmitting electrical stimulation received from said control unit to patient skin, from textile electrodes and the control unit to said textile electrodes and carrying the signal.

18. The electronic textile based rheumatoid arthritis therapy glove according to claim 17 further comprising: a textile electrode formed with handling of conductive yarns on polyester knitted fabric as a consequence of the optimization of computer assisted embroidery parameters.

19. The electronic textile based rheumatoid arthritis therapy glove according to claim 17, further comprising: at least one signal transmission line for which conductive yarn is used and which is used with embroidery on polyester textile surface.

20. The electronic textile based rheumatoid arthritis therapy glove according to claim 17, wherein said control unit comprises at least one easily assemblable and disassemblable snap fastener structure thanks to the snap fastener structure thereof.

21. The electronic textile based rheumatoid arthritis therapy glove according to claim 19, further comprising: a signal transmission line insulation located around said signal transmission line.

22. The electronic textile based rheumatoid arthritis therapy glove according to claim 21, wherein said signal transmission line insulation is comprised of thermoplastic membrane material.

23. The electronic textile based rheumatoid arthritis therapy glove according to claim 17, further comprising: joint compression and immobilization lamina enabling immobilization for treatment of swan-neck and buttonhole (boutonniere) deformations formed in finger joints caused by rheumatoid arthritis disease, and located such that it will run transversely in upper portion of second joints of the fingers and linearly in the rear portion thereof.

24. The electronic textile based rheumatoid arthritis therapy glove according to claim 17, further comprising: a plastic immobilization support used for treatment of ulnar deviation caused by rheumatoid arthritis disease.

25. The electronic textile based rheumatoid arthritis therapy glove according to claim 24, further comprising: an immobilization support pocket enabling positioning of said immobilization support on said glove.

26. The electronic textile based rheumatoid arthritis therapy glove according to claim 17, further comprising: a wristlet preventing ulnar deviation by starting from the end of little finger of the patient and being wrapped around the wrist portion of the glove, and enabling wrist stabilization.

27. The electronic textile based rheumatoid arthritis therapy glove according to claim 26, further comprising: a wristlet fixation apparatus enabling fixation of said wristlet.

28. The electronic textile based rheumatoid arthritis therapy glove according to claim 23, wherein said joint compression and immobilization lamina is comprised of thermoplastic polyurethane membrane material with compression feature.

29. The electronic textile based rheumatoid arthritis therapy glove according to claim 18, further comprising a plurality of textile electrodes being twelve in total, with six on the upper portion of said glove and six on the inner portion thereof.

30. The electronic textile based rheumatoid arthritis therapy glove according to claim 28, wherein the textile electrodes on the upper portion of said glove are positioned to correspond to the first joint of the fingers and wrist of the patient.

31. The electronic textile based rheumatoid arthritis therapy glove according to claim 28, wherein the textile electrodes on the inner portion of said glove are positioned to correspond to the second joint of the fingers and wrist of the patient.

32. The electronic textile based rheumatoid arthritis therapy glove according to claim 18, wherein said textile electrode is comprised of silver-covered polyamide conductive yarn material.

33. The electronic textile based rheumatoid arthritis therapy glove according to claim 18, wherein said textile electrode is formed as a result of embroidery of the silver-covered polyamide conductive yarn material with certain patterns, seam density and seam length as a consequence of computer assisted embroidery parameters optimization.

34. The electronic textile based rheumatoid arthritis therapy glove according to claim 17, wherein said signal transmission line is comprised of silver-covered conductive yarn material.

35. The electronic textile based rheumatoid arthritis therapy glove according to claim 17, wherein said signal transmission line is produced by embroidering the silver-covered polyamide conductive yarn material onto the textile surface as integrated with the electrodes.
